(19) United States
(12) Patent Application Publication
(10) Pub. No.: US 2024/0404649 A1
(43) Pub. Date: Dec. 5, 2024

(54) MACHINE-LEARNING FOUNDATION MODEL FOR GENERATING BIOPOLYMER EMBEDDINGS

(71) Applicant: Atomic AI, Inc., South San Francisco, CA (US)

(72) Inventors: Nicholas Ranieri BOYD, Berkeley, CA (US); Stephan Johannes EISMANN, Redwood City, CA (US); Raphael John LAMARRE TOWNSHEND, San Francisco, CA (US); Brent TOWNSHEND, Menlo Park, CA (US); Brandon Micheal ANDERSON, San Francisco, CA (US)

(73) Assignee: Atomic AI, Inc., South San Francisco, CA (US)

(21) Appl. No.: 18/733,699

(22) Filed: Jun. 4, 2024

Related U.S. Application Data
(60) Provisional application No. 63/506,349, filed on Jun. 5, 2023.
(60) Provisional application No. 63/609,696, filed on Dec. 13, 2023.

(51) Int. Cl. G16C 20/30; G16C 20/70

(52) U.S. Cl. CPC G16C 20/30; G16C 20/70

(57) ABSTRACT

A property of interest for a biopolymer is predicted using a combined model that includes a foundation model and a task-specific model. The foundation model is trained at least in part using chemical mapping data and generates embeddings from biopolymer sequences. The task-specific model takes an embedding generated by the foundation model for a sequence and generates a prediction of whether the corresponding biopolymer molecule has a target property.

### CROSS-REFERENCE TO RELATED APPLICATIONS

This application claims the benefit of U.S. Provisional Patent Application No. 63/506,349, filed Jun. 5, 2023, and U.S. Provisional Patent Application No. 63/609,696, filed Dec. 13, 2023, both of which are incorporated by reference.

### BACKGROUND

### 1. Technical Field

The subject matter described relates generally to machine-learning and, in particular, to a foundation model for generating biopolymer embeddings that can be applied to a wide range of property-prediction tasks.

### 2. Background Information

Various types of biopolymer are of great interest for therapeutic uses. RNA is currently of particular interest as a source of new therapeutics. However, it has proved extremely challenging to predict the properties of a particular RNA sequence. Even small changes in sequence can result in vastly different properties. Machine-learning provides a powerful tool for identifying RNA sequences that are likely to have a property of interest, but training models is difficult and computationally expensive. Furthermore, such models are often highly specialized to a specific task. For some tasks, training data is scarce, making accurate training of a model even more challenging.

A major challenge in the design of RNA-focused therapies is the lack of ground truth data to use for modeling. Functional data, such as on siRNA toxicity, can often only be collected at low throughput. With respect to structural data, few experimentally determined tertiary structures of RNA are available. In fact, only 1% of entries in the Protein Data Bank (PDB) comprise RNA alone, despite the over 10-fold excess of genome intervals that produce RNA relative to proteins. While evolutionary information encoded in multiple sequence alignments (MSAs) can provide critical insights on structure and function, these alignments are often shallow and uninformative for human targets and engineered sequences. Consequently, state-of-the-art RNA structure and function prediction approaches fall short of the recent successes of highly accurate protein prediction methods.

### SUMMARY

A method, computer-readable medium, and system enable the prediction of various properties of biopolymer (e.g., RNA) molecules from the corresponding sequence using machine learning. In one embodiment, the method is broken down into two portions. First, a high-capacity model is trained on a large dataset or collection of datasets related to biopolymers. Second, the pretrained model is combined with a task-specific model to improve the predictive performance for a task of interest (e.g., a model to predict whether an input sequence corresponds to a biopolymer molecule with a desired property). The initial large model is called a foundation model and the task of interest is called the downstream task. In other embodiments, different approaches to training may be used. For example, the training process may alternate between training the foundation model and the task-specific mode, or it may include one or more instances of training the combination of the foundation model and the task-specific model.

The disclosed approaches can provide significant improvements on a wide variety of downstream tasks, especially when data for the downstream task is limited. Many scientifically and commercially important prediction tasks for RNA molecules and other biopolymers fall into this category. In various embodiments, the foundation module is trained to generate an embedding of an input biopolymer sequence. Some or all of the training data may be chemical mapping data, which includes a significant amount of information about biopolymer structure, much of which is not immediately apparent to human observers. Thus, the embeddings generated by the trained model can include a lot of information about the properties of the corresponding biopolymer molecules. A small probe neural network added to the end of the foundation model can therefore be quickly trained with relatively little training data to extract the relevant structural information for a particular prediction task from the embeddings generated by the foundation model.

In one such embodiment, a computer-implemented method of predicting a target property of a biomolecule (e.g., RNA) includes obtaining first training data. The first training data includes first biopolymer sequences and corresponding experimentally obtained data. The method also includes training a foundation model using the first training data to predict the experimentally obtained data from the biopolymer sequences and adding a task-specific model to the foundation model to create a combined model. The method further includes training the combined model using second training data to predict the target property of biomolecules corresponding to second biopolymer sequences. The combined model may be applied to a previously unseen biopolymer sequence to generate a prediction of whether a candidate biomolecule corresponding to the previously unseen biopolymer sequence has the target property.

In various embodiments, adding the task specific model to the foundation model may involve removing zero or more output heads from the foundation model and adding one or more task-specific model heads. Training the combined model may involve freezing the layers of the foundation model such that just parameters of the task-specific model are modified. The target property may be secondary structure, tertiary structure, presence of a pocket with predetermined criteria, splicing activity, or whether the biomolecule will bind to a target molecule. The experimentally obtained data may include chemical mapping data.

### BRIEF DESCRIPTION OF THE DRAWINGS

FIG. 1 is a block diagram of a networked computing environment suitable for training and deployment of a foundation model for generating molecular embeddings, according to one embodiment.

FIG. 2 is a block diagram of the server of FIG. 1, according to one embodiment.

FIG. 3 is a flowchart of a method for training and using a foundation model, according to one embodiment.

FIG. 4 illustrates an example in which a simple linear model was trained on a single secondary structure and yields qualitatively-reasonable predictions of secondary structure for other input sequences, according to one embodiment.

FIGS. 5A-D illustrate the improved accuracy of secondary structure predictions generated using foundation model embeddings, according to one embodiment.

FIGS. 6A-C illustrate the improved accuracy of tertiary structure predictions generated using foundation model embeddings, according to one embodiment.

FIG. 7 is a comparison of tertiary structures generated using foundation model embeddings to the corresponding known structures for a set of example molecules, according to one embodiment.

FIGS. 8A and B illustrate the success of the foundation model when applied to the Stanford Open Vaccine Kaggle community prediction challenge, according to one embodiment.

FIG. 9 is a block diagram illustrating an example of a computer suitable for use in the networked computing environment of FIG. 1, according to one embodiment.

### DETAILED DESCRIPTION

The figures and the following description describe certain embodiments by way of illustration only. One skilled in the art will readily recognize from the following description that alternative embodiments of the structures and methods may be employed without departing from the principles described. Wherever practicable, similar or like reference numbers are used in the figures to indicate similar or like functionality. Where elements share a common numeral followed by a different letter, this indicates the elements are similar or identical. A reference to the numeral alone generally refers to any one or any combination of such elements, unless the context indicates otherwise.

### EXAMPLE SYSTEMS

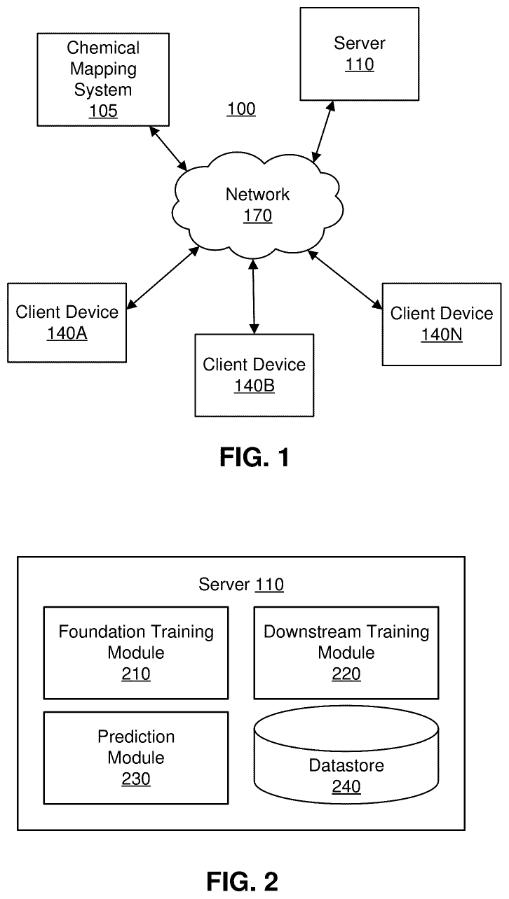

FIG. 1 illustrates one embodiment of a networked computing environment 100 suitable for training and deployment of a foundation model for generating molecular embeddings. In the embodiment shown, the networked computing environment 100 includes a chemical mapping system 105, a server 110, and client devices 140A, 140B, . . . , 140N, all connected via a network 170. In other embodiments, the networked computing environment 100 includes different or additional elements. In addition, the functions may be distributed among elements in a different manner than described. For example, the foundation model may be trained or used by a stand-alone system without reliance on a network.

The chemical mapping system 105 generates chemical mapping data for biopolymers (e.g., RNA). In chemical mapping, the biopolymer is exposed to a chemical agent that modifies (e.g., methylates, acylates, cross-links, attaches an adduct to, or digests) portions of the biopolymer. The chemical agent has different interactions with different parts of the biopolymer depending on the properties of the biopolymer (e.g., easily accessible portions of the biopolymer may interact more than shielded portions of the biopolymer). For example, the chemical agent is more likely to interact with unpaired nucleotides in RNA than paired nucleotides. Similarly, the chemical agent is more likely to interact with nucleotides on the outside of a folded RNA structure than those inside of it. Thus, if the RNA is sequenced after exposure to the chemical agent, the degree to which each nucleotide is impacted by the chemical agent contains information about the secondary and tertiary structure of the RNA.

In one embodiment, the chemical mapping system 105 includes apparatus for exposing RNA molecules to a chemical mapping agent such as by addition of a solution of DMS (Dimethyl Sulfide) to a tube containing RNA, a sequencing system (e.g., a next generation sequencing (NGS) system), and a database (or other datastore) for storing the generated sequencing data in conjunction with metadata (e.g., the pre-chemical exposure RNA sequence, chemical mapping conditions such as temperature, solution buffers, chemical mapping reagents, in cell vs. in vitro, cell type or source, etc.).

The server 110 includes one or more computing devices that train or apply one or more machine-learning models using experimentally gathered data regarding biopolymers. In one embodiment, the experimentally gathered data includes chemical mapping data. The chemical mapping data may include the rate of mutations in the sequencing readout at each nucleotide position compared to the original templates, the rate of termination of sequencing reads at each position, other per-nucleotide data, or per-sequence data, etc. The server 110 uses training data including the chemical mapping data to train a foundation model to generate embeddings from RNA sequences.

The server 110 appends one or more downstream task-specific models to the foundation model (e.g., by replacing the output layer of the trained foundation model with a multilayer perceptron or other small model). The server 110 trains the combined foundation and task-specific models on task-specific training data. Because the chemical mapping data (and thus the embeddings generated by the foundation model) contains information about the relationships between RNA sequence and properties such as tertiary structure of molecules with that sequence, the task-specific models can be trained with relatively few training examples to predict a wide range of properties, such as RNA secondary and tertiary structure, the presence and location binding site for another molecule (protein, RNA, DNA, or small molecule), the strength and selectivity of intermolecular binding, splicing activity, ribozyme activity, mRNA stability, IRES activity, or microRNA, siRNA, and ASO activity, etc. Alternatively, the foundation model and task-specific model may be trained together or training may alternate between the foundation model and the task-specific model until one or more criteria are met (e.g., a fixed number of iterations or achievement of a target accuracy on a validation set, etc.). Various embodiments of the server and models are described in greater detail below, with reference to FIG. 2.

The client devices 140 are computing devices with which a user my access functionality provided by the server. Although three client devices 140 are shown in FIG. 1, the networked computing environment 100 may include any number of such devices. In one embodiment, a client device 140 provides a user interface (e.g., in a web browser or dedicated software) via which the user can submit an RNA sequence to the server 110 in conjunction with a request to predict one or more properties of the molecule that forms from the RNA sequence. The server 110 applies one or more of the trained models to the sequence to generate the requested predictions and returns them to the client device 140 for display to the user. In another embodiment, the user may submit a request for an RNA sequence with one or more properties and parameters defining a range of sequences to consider and the server 110 may iterate through possible sequences (in accordance with the provided parameters) and provide one or more ranked results based on likelihoods of sequences having the requested property as determined by the trained model.

The network 170 provides the communication channels via which the other elements of the networked computing environment 100 communicate. The network 170 can include any combination of local area and wide area networks, using wired or wireless communication systems. In one embodiment, the network 170 uses standard communications technologies and protocols. For example, the network 170 can include communication links using technologies such as Ethernet, 802.11, worldwide interoperability for microwave access (WiMAX), 3G, 4G, 5G, code division multiple access (CDMA), digital subscriber line (DSL), etc. Examples of networking protocols used for communicating via the network 170 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 170 may be represented using any suitable format, such as hypertext markup language (HTML) or extensible markup language (XML). In some embodiments, some or all of the communication links of the network 170 may be encrypted using any suitable technique or techniques.

FIG. 2 illustrates one embodiment of the server 110. In the embodiment shown, the server 110 includes a foundation training module 210, a downstream training module 220, a prediction module 230, and a datastore 240. In other embodiments, the server 110 includes different or additional elements. In addition, the functions may be distributed among elements in a different manner than described. For example, although FIG. 2 shows a single entity providing foundation model training, downstream task-specific model training, and trained model application to generate predictions, each of these functions may be performed by a different device. These functions are described as being performed by a single entity for ease of explanation of the relevant concepts.

The foundation training module 210 trains one or more foundation models using training data that includes chemical mapping data. In one embodiment, a chemical mapping dataset includes a list of RNA sequences, each with an associated collection of reads. Each read is a sequence that may be identical to the original sequence or may contain any number of mutations relative to the original sequence (including point mutations, insertions, and deletions). Mathematically, this can be represented as:

$$𝒟 := ( {s}_{1} , ( {r}_{11} , … , {r}_{1 {l}_{1}} ) ) , … , ( {s}_{n} , ( {r}_{n 1} , … , {r}_{n {l}_{n}} ) )$$

Here, the dataset [symbol] includes chemical mapping data for n sequences s₁, . . . , sₙ where each sequence sᵢ has lᵢ reads rᵢ_{₁}, . . . , Tᵢₗ_{ᵢ}. Each sequence and read is a list of letters from the RNA alphabet: {A, C, G, U}. Because mutations are more likely to occur at some locations than others (e.g., paired nucleotides are less likely to mutate than unpaired nucleotides, while mutations are more common on the exterior of the tertiary structure of the RNA than for interior nucleotides), the probability distribution of mutations inherently includes information about the structure of the RNA molecule.

The foundation model may be trained on a collection of multiple chemical mapping datasets, each collected under varying experimental conditions on the same or different sequences. The different experimental conditions may include one or more of variable temperatures, different solution buffers, different chemical mapping reagents, in cell vs. in vitro, cell type, source of cells, replicates of previously used conditions, etc. For example, the training data may include chemical mapping data for a first group of one or more sequences at each of a first set of (e.g., five) temperatures and a second group of one or more sequences at each of a second set of (e.g., two) temperatures, which may be a subset of the first set of temperatures or different temperatures. Training the model on diverse datasets can improve its ability to generalize to new downstream tasks. For simplicity, the following description describes training the foundation model with a single chemical mapping dataset but it should be appreciated that any number of chemical mapping datasets may be used by applying the same principles.

The foundation model may be defined by a model class and is trained by attempting to minimize a loss function. The model class is a parametric function class [symbol] where each function ƒ_{θ}∈ [symbol] is parameterized by a vector of numbers θ. The vector may be very large, e.g., having over one million, over ten million, over one hundred million, over one billion, or more values. In one embodiment, [symbol] is chosen such that each ƒ_{θ} maps an RNA sequence to a distribution over possible reads. To be precise, ƒ_{θ}: {A, U, C, G}*→ [symbol] ({A, U, C, G}*) where {A, U, C, G}* is the set of all RNA sequences and [symbol] ({A, U, C, G}*) is the set of probability distributions over RNA sequences. A particular model ƒ_{θ} can be thought of as a simulator for the chemical mapping experiment that generated the dataset, thus enabling predictions of the distributions over reads for novel RNA sequences.

The loss function [symbol] maps an estimated distribution over reads, \hat{p}, and a collection of reads r₁, . . . , rₗ to a single number which measures how bad the prediction \hat{p} is for those reads. In one embodiment, [symbol] is the negative log likelihood of the reads under the estimated distribution. The loss function may be modified to be subject to simplification or data-dependent scaling to reduce the computational requirements for training, improving the overall efficiency of the model.

Because the observed reads are often dominated by simple point mutations (instead of deletions or insertions), the mutations within each read are approximately independent, and the type of mutation (e.g., A→C) does not contain much information, the model may be substantially simplified. In one embodiment, the simplification first involves aligning all reads to the input sequence and discarding reads with insertions or deletions or ignoring a limited number of insertions or deletions. This leaves a collection of reads that are all the same length as the input sequence. The dataset can then be simplified (after removing reads that are not simple point mutations) to a collection of mutation counts and total counts for each position in each input sequence. A mutation count is the number of reads that have a mutation at that position and a total count is the total number of reads at that position. In another embodiment, the insertions or deletions may also be included in the mutation counts.

Instead of predicting a distribution over all possible reads, a marginal mutation probability can be predicted for each position in the input sequence. Note that this is equivalent to a product distribution over all reads of the same length as the input sequence (with a uniform distribution over the three possible mutations for each position). The loss function can then be simplified to a sum of binomial losses, one for each position in each sequence. The binomial loss is the negative log likelihood of the observed mutation count given the total count and the predicted mutation probability.

Representing this mathematically, for an input sequence s of length n, the model predicts n numbers p₁, . . . , pₙ, each between 0 and 1, and the loss function is:

$$ℒ ( p , m , c ) := - {\sum }_{i = 1}^{n} log ( B ( {m}_{i} , {c}_{i} , {p}_{i} ) )$$

where B(m, t, p) is the probability mass function of the binomial distribution with parameters m, c, p, mᵢ is the mutation count at position i, and cᵢ is the total count at position i. The computational savings from this simplification can be substantial. At the very least they reduce the amount of training data by a factor of the average number of reads per sequence (which can be in the thousands).

Because each sequence in the dataset has a different number of reads, the loss function is not directly comparable between sequences. This imbalance can cause issues during training and with downstream performance. This problem may be mitigated or solved using data-dependent scaling. In one embodiment, the data-dependent scaling includes dividing the loss for each training example by min (Σᵢcᵢ, T) where Tis a minimum count threshold (e.g., T=500). This ensures that the loss for each sequence is approximately on the same scale, except for sequences with very few reads.

Having described the model class and loss function, it should be appreciated that a range of model architectures may be used. In one embodiment, for training purposes, the foundation model includes three parts: (1) a sequence embedder; (2) a trunk; and (3) one or more output heads (e.g., one for each chemical mapping experiment).

The sequence embedder and trunk each produce one or more embeddings (or internal representations) of an input sequence. In one embodiment, an embedding is a tuple of two arrays: a single representation and a pair representation. For a sequence of length n, the single representation is an array of size (n, d) and the pair representation is an array of size (n, n, d′). Each (internal) layer in the network takes an embedding as input and produces an embedding (with the same dimensions) as output. Intuitively the single representation contains information about each nucleotide in the sequence, while the pair representation encodes the interactions between pairs of nucleotides.

The sequence embedder turns an RNA sequence of length n into an initial embedding. For the single representation each nucleotide may be encoded as a one-hot vector of length four and then passed through a linear layer to get a vector of size d for each nucleotide. For each pair of nucleotides (nᵢ, nⱼ), the relative displacement between them may be encoded as a one-hot vector of predetermined length (e.g., 65) by clipping j-i to a corresponding range (e.g., [−32, 32] for a predetermined length of 65). This one-hot vector can then be concatenated with the length eight vector of the one-hot encodings of nᵢ and nⱼ and passed through a linear layer to get a vector of size d′ for each pair of nucleotides.

The trunk takes as input the initial embedding produced by the sequence embedder, refines it using a series of one or more layers, and produces an embedding of the same size as output. Each trunk layer takes an embedding (either the initial embedding or the embedding generated by a previous trunk layer) as input and produces an embedding as output. In one embodiment, each trunk layer is made up of an identical set of sublayers but has different parameters. If the input to a trunk layer is the embedding (s, P), then the trunk layer performs the following operations:
1) Pass the single representation and the pair representation through two LayerNorm sublayers;
2) Concatenate the normalized pair representation with reshaped versions of the normalized single representation to form a three-dimensional array T: Tᵢⱼ=(Pᵢⱼ, sᵢ, sⱼ);
3) Pass each slice of T (along the final dimension) through a two-layer multi-layer perceptron to produce T′;
4) Split T′ along the last dimension into three arrays: α of shape (n, n, 1), M with shape (n, n, d), and E with shape (n, n, d′);
5) Compute a row-wise softmax of a to form the n by n matrix w;
6) Compute the node messages Nᵢ=ΣⱼWᵢⱼMᵢⱼ;
7) Concatenate N with the normalized single representation s and pass it through a two-layer convolutional network with kernel size 3 and GELU nonlinearities;
8) Add the resulting array to the unnormalized single representation;
9) Update the unnormalized pair representation by summing it with E; and
10) Apply triangle layers to the pair representation, e.g., applying (in series) two layers of triangle self-attention and two triangle-multiplicative updates in residual fashion—the use of triangle layers can substantially improve performance.

The output heads take the embedding produced by the trunk as input and produce a prediction for the mutation probability at each position in the input sequence. If the input to an output head is the embedding (s, P), the output head predicts a mutation probability pᵢ for each position i in the input sequence by applying a linear layer followed by a sigmoid nonlinearity to sᵢ to produce a single real number between 0 and 1.

The foundation training module 210 can use any appropriate training algorithm. In one embodiment, the foundation training module 210 uses the AdamW optimizer with weight decay and a linear learning rate warmup to a peak learning rate of 5E⁻⁴ and cosine decay to zero over seven million steps. The training may be stabilized with gradient clipping. Training may be performed using multiple (e.g., eight) NVIDIA A100 GPUs in parallel and a predetermined batchsize (e.g., a batchsize of eight). To improve downstream performance, the foundation training module 210 may save a running exponential weighted average of the model parameters with α=0.999.

The downstream training module 220 starts with a trained foundation model and adds a task-specific model to produce a combined model. The combined model may be created by removing the output head(s) and replacing them with the task-specific model. Thus, the task specific model receives the embedding generated by the last layer in the trunk of the foundation model as input.

The downstream training module 220 uses task-specific training data (e.g., sequences labeled with whether the corresponding molecule has a target property) to train the combined model. The foundation model may be frozen during training of the combined model, such that only parameters of the task-specific model can be modified. Thus, the combined model may be trained with relatively little training data as the foundation model is already trained to extract pertinent information from the input sequence and represent it in the embedding that is provided to the task-specific model. Alternatively, the foundation model may be retrained (or trained from scratch) in parallel with training of the task-specific model.

In one example embodiment, the task-specific model is trained to predict the secondary structure formed from an RNA sequence. A secondary structure is a set of Watson-Crick-Franklin base pairs {i, j} such that each index i only appears in one pair. Often the secondary structure is represented as a matching matrix: a symmetric matrix with entries in {0, 1} such that every row and column has a single one. The entry (i, j) is 1 if and only if i and j are paired and (i, i) is 1 if and only if i is unpaired. A secondary structure dataset is then a list of pairs (s, M) where s is an RNA sequence and M is a matching matrix for s.

Using the trained foundation model as a starting point, the downstream training model 220 can use a simple probe (a small model fit on top of the foundation model embeddings) to predict the matching matrix. To be precise, this means using the pretrained foundation model to compute the embedding at the end of the trunk and using a small model to predict the matching matrix from this embedding. Because the foundation model embeddings contain substantial information about secondary structure, the probe can be trained with as few as one training example. For example, a linear model may be used to predict the (i, j) entry of the matching matrix directly from the corresponding entry of the pair representation Pi,j. This model is very simple and has only d′ parameters. Fitting this model with a least-squares loss function leads to substantially accurate predictions when trained on a single sequence (e.g., (corresponding to Protein Data Bank entry 1GID) and matching matrix pair. For example, testing the approach and model on 38 single-stranded RNAs from the PDB, the average F1 score on base pair prediction was 0.8 (comparing the predicted base pairs with those identified by the program DSSR based on the experimentally determined structures in the PDB). Specifically, the model was tested on: 2K96, 2NBZ, 6W3M, 5V17, 5KH8, 6MXQ, 2N4L, 6NOA, 6 VAR, 2NC1, 2N8V, 2N7M, 7LVA, 6UES, 2N1Q, 5MOH, 6D3P, 3NPQ, 4ENC, 6TFF, 5BTM, 4XWF, 4PQV, 5OB3, 3IVN, 4TZX, 5KPY, 2OIU, 3D2G, 6UGG, 4FRG, 3RG5, 5T83, 4L81, 1Z43, 6WJR, 6OL3, and 1U9S. Using a slightly more sophisticated model (e.g., a two-layer MLP), training on more examples, and using a binary cross-entropy loss can produce an estimator that is competitive with existing state of the art methods (e.g., RNAFold).

In another example embodiment, the task-specific model is trained to predict the results of an RNA small molecule binding assay. To simplify the problem, the measurement can be treated as binary—either the molecule binds with a minimum affinity or it does not—but the model may also be trained to provide predictions of nonbinary measurements (e.g., a binding affinity). Assuming binary measurements, a dataset for this task includes of a list of tuples (s₁, m₁, b₁), . . . , (sₙ, mₙ, bₙ) where sᵢ are RNA sequences, mᵢ are small molecules, and bᵢ∈{0, 1} are binary labels for binding/non-binding.

For this task, the downstream training module 220 model applies a model that first computes descriptors of both the RNA sequence s and the small molecule m and then uses a simple MLP to predict the binding probability p from the concatenation of the descriptors (although more sophisticated architectures are of course possible). To compute the structure descriptor, the combined model first computes the embedding at the end of the trunk (s, P) of the foundation model and then processes the embedding by running it through a new, trainable instantiation of a trunk network (which may use different hyperparameters, e.g., depth and width, than the original pretrained trunk). The processed single representation s′ produced by this adapter network is used to compute a mean over the first dimension, which produces a single vector of length d, the structure descriptor.

The small molecule descriptor may be computed using any suitable technique, such as by using Mordred (Moriwaki, Hirotomo, et al., “Mordred: a molecular descriptor calculator.” Journal of cheminformatics 10.1 2018:1-14). Mordred produces a descriptor vector in R¹⁶¹³. The binding probability may be computed by concatenating the structure descriptor and the molecule descriptor and passing the result through a three-layer MLP with a single output unit and a sigmoid nonlinearity. AdamW may be used for training with a linear warmup and cosine decay learning rate schedule, gradient clipping, and a binary cross-entropy loss.

In another example embodiment, the task-specific model is trained to jointly predict three per-nucleotide statistics: reactivity, degradation rate in the presence of magnesium at high pH, and degradation rate in the presence of magnesium at high temperature. The training data includes measurements taken from 2400 107-nucleotide mRNA sequences originating from the Eterna Roll-Own-Structure-Competition. Measured properties are provided for the first 68 nucleotides per sequence in this training set. A dataset for this task consists of a list of RNA sequences and three real values for the first 68 nucleotides.

The sequence is then passed through the embedding and trunk modules of a model pre-trained with chemical mapping data to obtain a single and pair representation (of sizes 107×512 and 107×107×256, respectively). For the purpose of the task-specific model, there are no trainable parameters in this first part of the neural network architecture. The single representation is first linearly projected down to 64 dimensions (107×64) and then passed through, and subsequently manipulated through, 3 ‘PTransformer’ blocks, with no shared weights between layers. Each Ptransformer (transformer from here on) layer is a variation on a standard transformer module, with the variation being that the self-attention weights aᵢⱼ are calculated by passing the pair representation between the i-th and j-th nucleotides through a shallow multi-layer perceptron. The result of this process is a new 107×64 single representation.

The model output is obtained by projecting the transformed single representation down to L×3. The model is trained using the AdamW optimizer, with a cosine annealing learning rate schedule and gradient clipping. The model is trained to optimize the MCRMSE loss, with the modification that the per-nucleotide loss is re-weighed according to error estimates provided in the training dataset for the different target values, specifically the per-nucleotide loss is re-weighted according to ½+exp (−5*E(nucleotide,target)) where E(nucleotide,target) is the per-nucleotide-per-target error estimate.

When evaluated on a public test set from the Open Vaccine Kaggel challenge, accuracy (as measured by Root Mean Squared Error (RMSE) and R-squared) is starkly improved when comparing model performance with and without pretraining with chemical mapping data. For the test set, which consists of around 600 107-nucleotide sequences with target values to predict for the first 68 nucleotides, average RMSE across tasks with and without pretraining is 0.24 and 0.31, respectively. R-squared increases from 0.49 to 0.72 comparing models without and with chemical mapping data for pretraining.

In some embodiments, the foundation training module 210 trains multiple foundation models using different training data. The downstream task-specific models may be configured to take the embeddings generated by an ensemble (some or all) of the trained foundation models as input and generate a prediction of whether the RNA molecule corresponding to the input sequence has the target property.

The prediction module 230 provides a user interface (e.g., to a client device 140) via which trained combined model models can be applied to new sequences. In one embodiment, a user selects one or more target properties (e.g., from a library of target properties for which models have been trained) and provides an RNA sequence and the prediction module 230 applies one or more models to generate corresponding predictions of whether the RNA sequence corresponds to a molecule with the target properties. If multiple target properties are selected, the prediction module 230 may apply multiple models (e.g., one for each target property), apply a multiplexed model (i.e., one that is trained to predict multiple properties from an input sequence), or use a combination of both approaches.

The datastore 240 includes one or more non-transitory computer-readable media configured to store the data and models used by the server 110. For example, the datastore 240 can include one or more hard drives that store the trained models generated by the foundation training module 210 and downstream training module 220. The datastore 240 may also include the training data used to train models. Although the datastore 240 is shown as a single entity within the server 110, the data and models may be spread across multiple devices at multiple locations (e.g., in a distributed database accessed via the network170).

### EXAMPLE METHODS

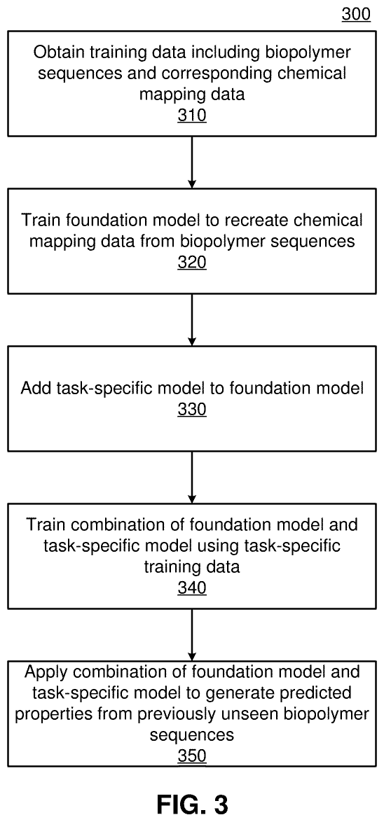

FIG. 3 illustrates an example method 300 for training and using a foundation model, according to one embodiment. The steps of FIG. 3 are illustrated from the perspective of the server 110 performing the method 300. However, some or all of the steps may be performed by other entities or components. In addition, some embodiments may perform the steps in parallel, perform the steps in different orders, or perform different steps. For example, although single instances of training the foundation model and combined model are shown as distinct steps, training may alternate between training the foundation and combined models iteratively.

In the embodiment shown in FIG. 3, the method 300 begins with the server 110 obtaining 310 training data. The training data includes biopolymer (e.g., RNA) sequences and corresponding chemical mapping data. The server 110 trains 320 a foundation model to predict (e.g., recreate) the chemical mapping data from the biopolymer sequences. As described previously, the layer preceding the output heads in the foundation model includes an embedding of the input sequence that includes information regarding the structure of the corresponding molecule.

The server 110 adds 330 a task-specific model to the foundation model to create a combined model. The task-specific model is configured to predict a particular property of the molecule corresponding to an input sequence. Adding 330 the task-specific model may include removing the output head or heads from the foundation model and replacing them with one or more layers of the task-specific model. The server 110 trains 340 the combined model using task-specific training data. Because the foundation model has already been turned to generate embeddings, the task-specific model may be trained efficiently with relatively little training data. Furthermore, different task-specific models may be appended (either to the same or different instances of the foundation model) to enable prediction of a wide range of properties from an input sequence.

Once the combined model has been trained, it can be deployed to make predictions for whether previously unsees sequences have the property or properties for which it was trained. The server 110 may receive an input sequence and apply 350 the combined model to generate the predicted property or properties of the molecule corresponding to the input sequence.

### Example Implementations and Use Cases

What follows are specific details of the training and use of a foundation model for various exemplary use cases, according to various embodiments. These examples are included for illustrative purposes to provide teaching regarding the broader principles described above and should not be considered limiting. Rather, they demonstrate the broad functionality for probing properties of biopolymer molecules that is enabled by the disclosed foundation model and related techniques.

Training a Foundation Model with Chemical Mapping Data

Chemical mapping experiments modify RNA and produce a collection of sequencing reads for each input RNA species. Each read may include one or more substitutions, insertions, or deletions relative to the original sequence. As described previously, the distribution of these mutations is related to the structure (or ensemble of structures) of the input RNA, with different chemical mapping reagents and experimental conditions measuring different aspects of RNA structure. For many of these reagents, a first-order approximation is that unpaired nucleotides are more likely to result in mutations than paired nucleotides.

From a machine learning perspective this is a sequence-to-sequence problem: the input sequence is the RNA species, while the output sequences are the observed reads assigned to that species. Readout via NGS allows the input species to be multiplexed and experiments to be scaled to produce a large number (e.g., hundreds of billions) of tokens to train a high-capacity foundation model.

Chemical mapping data was collected using several chemical reagents on a set of diverse, custom-designed libraries under several different conditions. This data was used to train a foundation model using the neural network architecture of the sequence-to-sequence-to-sequence transformer-based model and approaches described above. For an RNA sequence of length n, the embedding produced by the encoder is two objects: the single representation, which is an array of size n-by-512, and the pair representation, an array of size n-by-n-by-256. In the following sections we show that the encoder's embeddings contain rich and accessible information on RNA structure and function.

Probe networks can be used to demonstrate the emergence of accurate and accessible representations in large, pretrained models. Computational probing experiments emulate the process of prototyping the use of the foundation model for a new prediction task. A typical probing experiment consists of two steps. First, a small network (the probe) is trained to predict the property of interest directly from the foundation model embeddings. Next, to show that performance of the probe is the direct result of the foundation model and not the training procedure or probe network, the same network is trained without access to embeddings (the baseline). If the performance of the probe when used with the embeddings is substantially better than that of the baseline, then it can be concluded that the foundation model contains useful and accessible representations of the property of interest.

### Secondary Structure Prediction

RNA secondary structure is characterized by patterns of hydrogen bonding between nucleotide bases in canonical Watson-Crick or wobble base pairs. These structures govern RNA's biological function and the design of RNA-focused therapies involves understanding relationships between secondary structure and biological impact. From a mathematical standpoint, a secondary structure S of an RNA of length n is a set of unordered pairs {i, j} where i≠j∈1, . . . , n. Each pair in S is called a base pair.

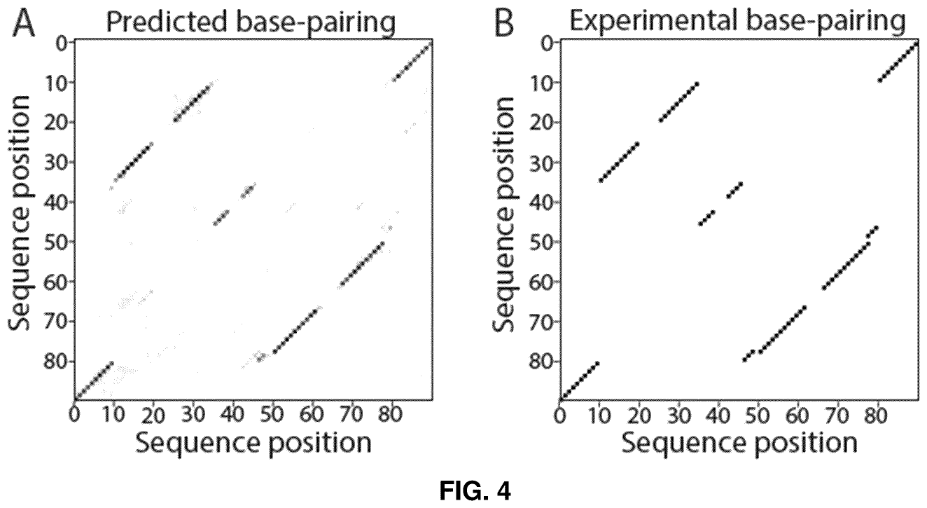

To evaluate the accuracy of the secondary structure representations developed by the model, embeddings generated by the foundation model were provided to probe networks. As base pairing is a property of each pair of nucleotides, it is natural to apply these probes to the pair representation independently along the last dimension. FIG. 4 illustrates an example in which a 257-parameter linear model was trained on a single secondary structure and yields qualitatively-reasonable predictions of secondary structure. In fact, despite only being trained on an FMN riboswitch aptamer structure (PDB ID: 6WJR, 112 nucleotides), this simple probe is able to generalize to distinct RNA classes, for instance a cloverleaf-like RNA domain (PDB ID: 8DP3, 90 nucleotides). Part A, on the left of FIG. 4, illustrates the predicted probability of each base pair for PDB ID 8DP3 estimated by the 257-parameter probe. Part B, on the right of FIG. 4, sows the ground truth secondary structure for PDB ID 8DP3 represented as a symmetric matrix of base pairs. This demonstrates that in the process of learning to predict chemical mapping data, the foundation model has developed an accessible representation of secondary structure.

To show that the secondary structure representations developed by the model are highly accurate, a slightly more expressive probe was also tested. This probe was a multilayer perceptron (MLP) with a single hidden layer of dimension 2048 (for a total of ˜2.6M parameters). In comparison, a probe was tested with the same architecture applied to RNA-FM, a foundation model trained on naturally-occurring RNA sequences. A baseline network with the same architecture applied described above was also applied only to sequence features.

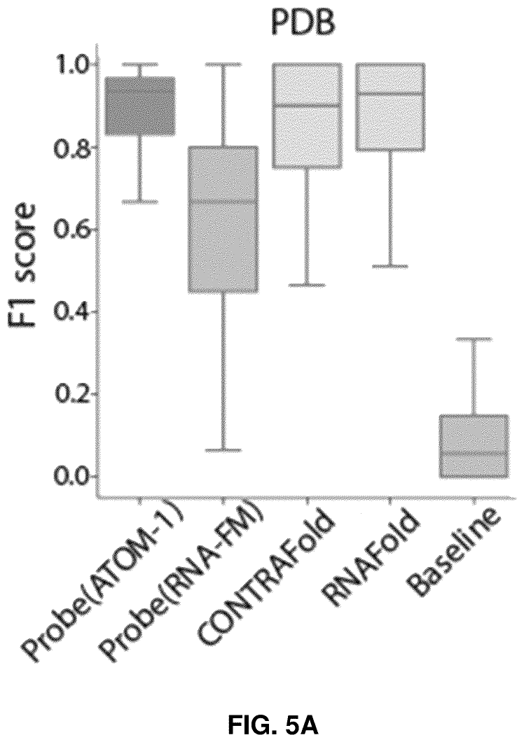

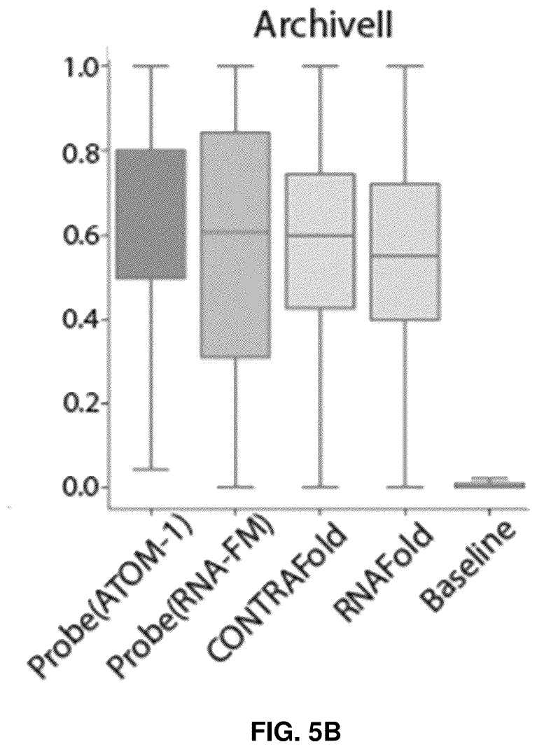

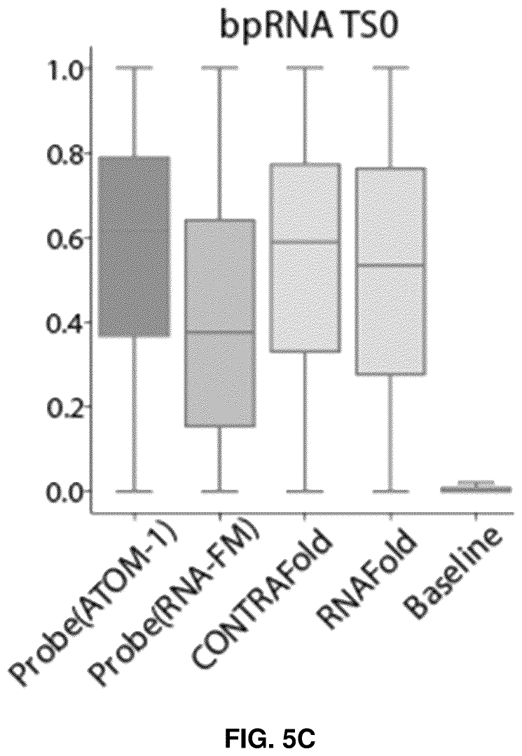

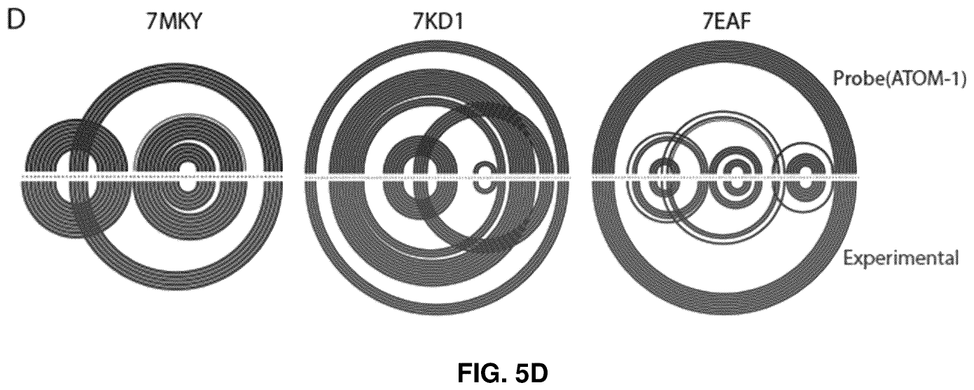

These probe networks were trained on a subset of single-chain RNA secondary structures derived from PDB entries from before Apr. 30, 2020. For testing, the trained probes were applied to secondary structures from PDB entries published after May 1, 2020, excluding sequences with more than 80% sequence identity to the training set from the evaluation. FIG. 5A presents the accuracies of the different prediction methods as measured by F1-score. The probe is competitive with physics-based methods, RNAFold and CONTRAFold, and performs substantially better than the same probe architecture applied to RNA-FM. The baseline—the probe architecture applied directly to sequence features—demonstrates minimal prediction accuracy.

To test the generalization capability of the probe, it was validated on two additional datasets: ArchiveII and bpRNA-1M-TS0. As with the PDB evaluation set, test cases with high sequence identity to the training set were removed. Secondary structure in these datasets is not derived from experimentally-determined tertiary structure, but inferred from multiple-sequence alignments. Despite the shift in domain, the probe remained highly accurate, demonstrating strong generalizationability. FIG. 5B illustrates the results for the ArchiveII dataset and FIG. 5C illustrates the results for the bpRNA-1M-TSO dataset.

The results demonstrate the broader finding that the probe generates accurate predictions for complex RNAs across diverse RNA classes and lengths, as illustrated in FIG. 5D. For instance, the probe was found to accurately predict secondary structures for a SARS-CoV-2 frameshift stimulation element construct, an apo THR riboswitch aptamer, and a SAM-I riboswitch variant. These examples demonstrate that the probe is able to correctly predict pseudoknots, secondary structure elements which physics-based methods often fail to predict.

Finally, it is notable that the probe technique used was purely local: each prediction for a pair of residues used only the single and pairwise representation for those two residues. This is in contrast to previous secondary structure techniques which use non-local dynamic programming algorithms, repeated convolutional layers with large receptive fields, or both. Because the probe network need not include any interactions between nucleotides (although some embodiments may include data representing such interactions), the predictive performance in these examples originates from the representation present in the foundation model embeddings alone.

### Tertiary Structure Prediction

While secondary structure is an important aspect of RNA, many therapeutically-relevant properties of RNA are mediated by the full tertiary (3D) structure. A natural question, then, is to what extent the foundation model contains readily-accessible 3D structural information, especially since one might suspect that chemical mapping data is dependent only on secondary structure. To answer this, the foundation model was probed using a shallow (two-layer), MSA-free variant of the Evoformer with a custom structure module. The model was trained and evaluated on RNA structures from the PDB.

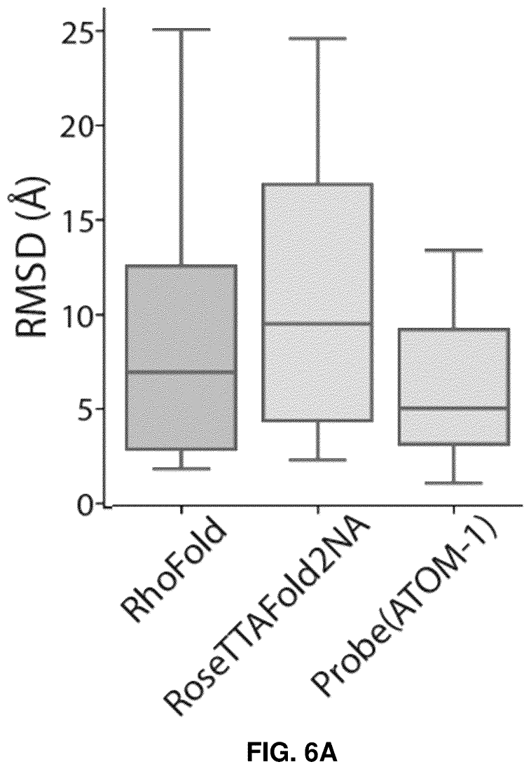

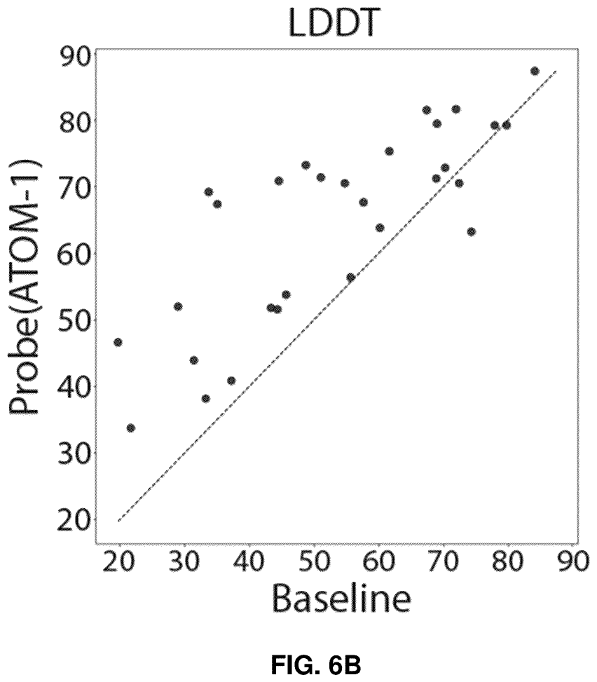

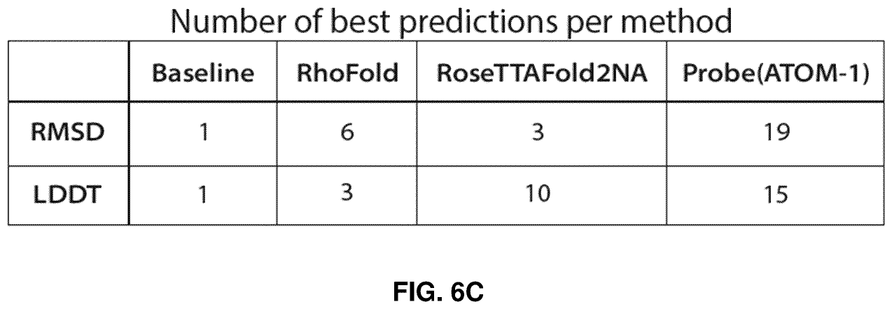

FIG. 6A compares the results from probing the foundation model to two state-of-the-art 3D structure prediction methods: RhoFold, the deep learning method with the best performance from CASP15, and RoseTTAFold2NA. Notably, both RhoFold and RoseTTAFold2NA make use of MSAs which are time-consuming to generate and are often unavailable for RNAs of interest. Despite having no access to MSAs and being considerably smaller (˜15M parameters) and shallower (2 layers) than RhoFold (˜100M parameters in 12 layers) and RoseTTAFold2NA (˜68M parameters in 40 layers), the combined foundation model and probe produced predictions with higher global accuracy as measured by root mean-squared deviation (RMSD).

FIG. 6B illustrates a comparison of the results obtained by probing the foundation model with a baseline network, which uses an identical architecture without the foundation model embeddings. Compared to the baseline network, the probe produced predictions with consistently higher local accuracy as measured by the local distance difference test (LDDT).

FIG. 6C illustrates that the probe generated the best 3D structure predictions more often than state-of-the-art deep learning methods and our baseline model based on both RMSD and LDDT. Together, these comparisons show that the foundation model produces readily accessible and accurate representations of RNA 3D structure.

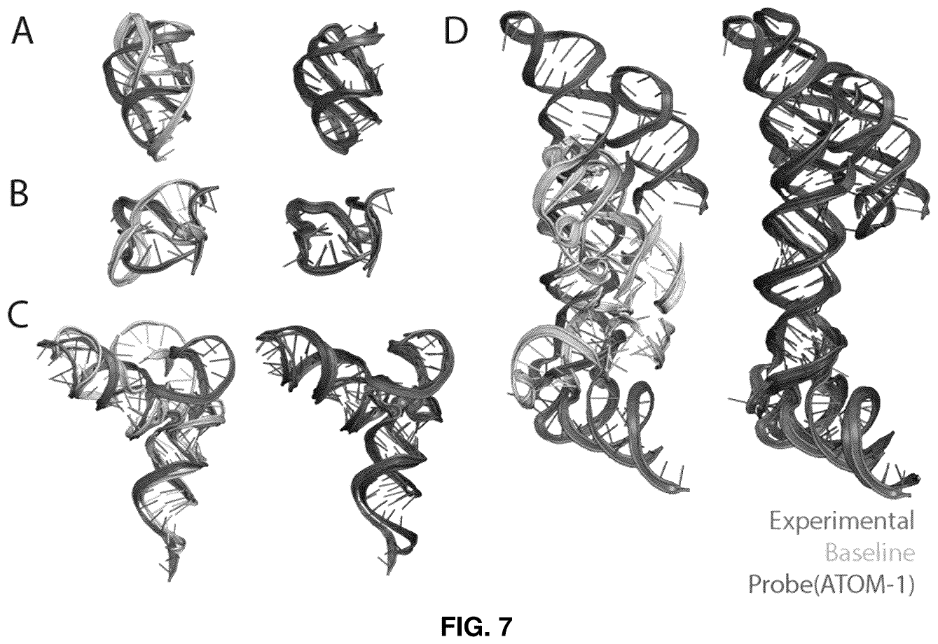

The utility of foundation model embeddings can be further demonstrated by generating visualizations of the predicted 3D structures generated using the embeddings. FIG. 7 shows some example 3D structures generated using the foundation model embeddings. Specifically, FIG. 7 shows predictions overlaid on experimental structures for: (A) a Pre-Q1 riboswitch (PDB ID: 8FB3); (B) a G-quadruplex (PDB ID: 7SXP); (C) a synthetic tRNA (PDB ID); and (D) a cloverleaf RNA fused with a tRNA (PDB ID: 8S95). The probe network applied to the foundation model embeddings produced RNA models that match the native global fold for diverse RNA targets across a broad range of sequence lengths. These predictions substantially outperformed the baseline model that did not use the foundation model embeddings. Notably, this improvement is apparent even in cases where the native structure includes mostly non-canonical base-pairing (for instance, the G-quadruplex), demonstrating that the foundation model embeddings contain structural information beyond secondary structure.

### In-Solution Stability

Successful distribution of mRNA vaccines requires mRNA constructs that are stable over long periods of time in solution. The ability of the foundation model to help predict RNA stability was evaluated using data from the Stanford OpenVaccine Kaggle community prediction challenge. A simple probe network (˜10M parameters) was trained to predict degradation and reactivity characteristics from the embeddings of the foundation model.

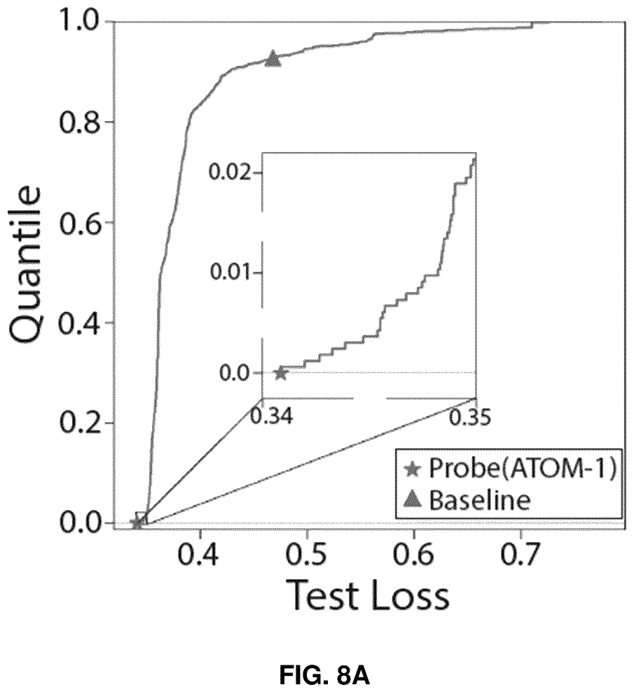

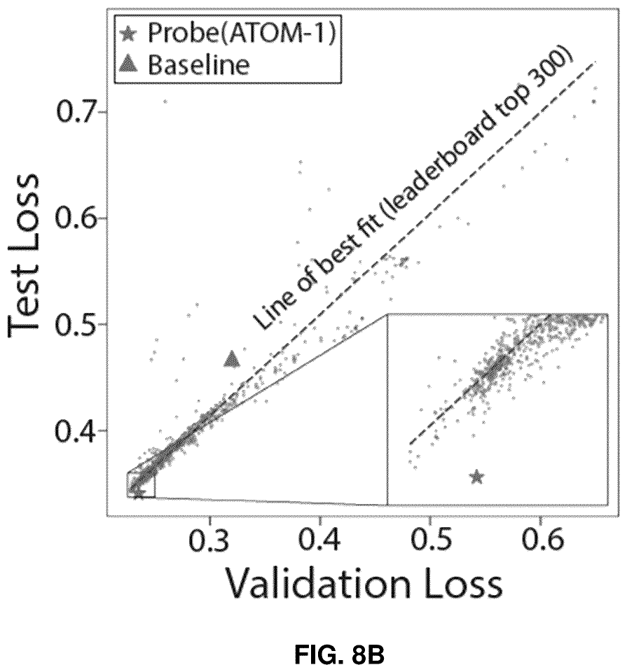

FIG. 8A illustrates that the foundation model and simple probe network outperformed all 1636 challenge submissions. For comparison, FIG. 8A also includes the accuracy of a baseline network without access to the foundation model embeddings but otherwise having the same architecture. The quantile value denotes the fraction of submissions with smaller (better) test losses. Lower quantile values indicate better performance. As in previous tasks, significant accuracy regression is observed—the test loss of the baseline network is 37% higher compared to the foundation model probe—indicating that the high prediction accuracy of the probe of the foundation model is not driven by the probe architecture or training procedure, but rather by structural information captured in the foundation model embeddings.

The design of this challenge showcases the generalization abilities of models built on top of the foundation model. FIG. 8B compares validation and test losses for the different methods that participated in the challenge. Lower values are better, with the black dashed line being a line of best fit on the top 300 submissions by test loss. Loss was calculated as the mean prediction RMSE across multiple prediction tasks. Note that the foundation model probe does particularly well with respect to the sequences in the test set, which are about 30% longer than those in the training and validation sets used. During the challenge, participants were able to repeatedly evaluate the accuracy of their methods on the validation set, likely leading to overfitting to this validation set by some methods, whereas an evaluation on the test set was not available until the end of the challenge.

Furthermore, it is notable that the foundation model was not pretrained or self-distilled using test set sequences, whereas the top Kaggle solutions used one or both of these approaches. While these methods are perfectly valid within the confines of the challenge, they are likely to lead to test metrics that are overly optimistic with respect to the prospective performance of models on new sequences-even those drawn from the same distribution as the test set.

### Computing System Architecture

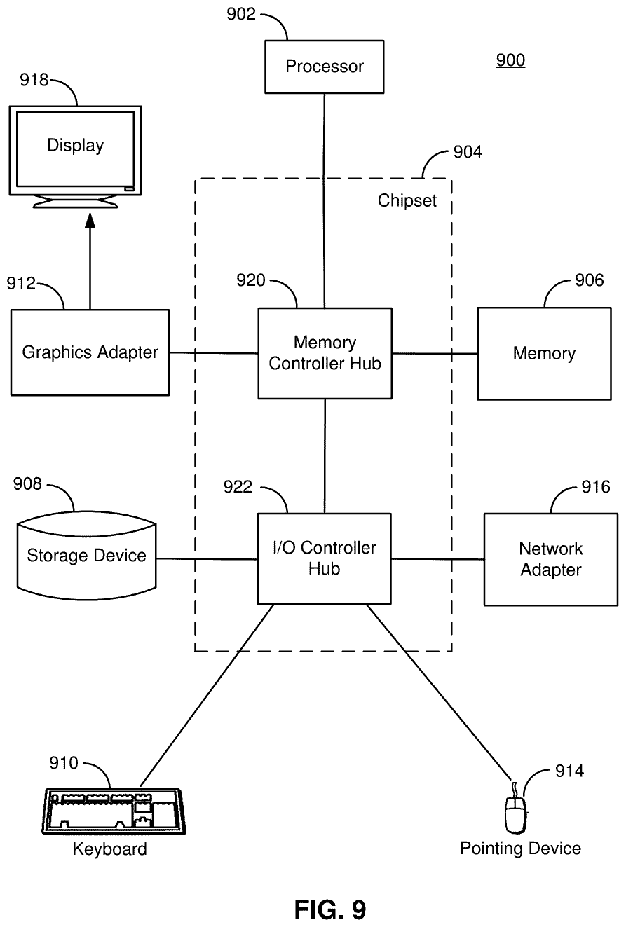

FIG. 9 is a block diagram of an example computer 900 suitable for use in the networked computing environment 100 of FIG. 1. The example computer 900 includes at least one processor 902 coupled to a chipset 904. The chipset 904 includes a memory controller hub 920 and an input/output (I/O) controller hub 922. A memory 906 and a graphics adapter 912 are coupled to the memory controller hub 920, and a display 918 is coupled to the graphics adapter 912. A storage device 908, keyboard 910, pointing device 914, and network adapter 916 are coupled to the I/O controller hub 922. Other embodiments of the computer 900 have different architectures.

In the embodiment shown in FIG. 9, the storage device 908 is a non-transitory computer-readable storage medium such as a hard drive, compact disk read-only memory (CD-ROM), DVD, or a solid-state memory device. The memory 906 holds instructions and data used by the processor 902. The pointing device 914 is a mouse, track ball, touchscreen, or other type of pointing device, and may be used in combination with the keyboard 910 (which may be an on-screen keyboard) to input data into the computer system 900. The graphics adapter 912 displays images and other information on the display 918. The network adapter 916 couples the computer system 900 to one or more computer networks, such as network 170.

The types of computers used by the entities of FIGS. 1 and 2 can vary depending upon the embodiment and the processing power required by the entity. For example, the server 110 might include multiple blade servers working together to provide the functionality described. Furthermore, the computers can lack some of the components described above, such as keyboards 910, graphics adapters 912, and displays 918.

### Additional Considerations

Some portions of above description describe the embodiments in terms of algorithmic processes or operations. These algorithmic descriptions and representations are commonly used by those skilled in the computing arts to convey the substance of their work effectively to others skilled in the art. These operations, while described functionally, computationally, or logically, are understood to be implemented by computer programs comprising instructions for execution by a processor or equivalent electrical circuits, microcode, or the like. Furthermore, it has also proven convenient at times, to refer to these arrangements of functional operations as modules, without loss of generality.

Any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the elements or components are present unless it is obvious that it is meant otherwise.

Where values are described as “approximate” or “substantially” (or their derivatives), such values should be construed as accurate +/−10% unless another meaning is apparent from the context. From example, “approximately ten” should be understood to mean “in a range from nine to eleven.”

The terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs for a system and a process training and using a foundation model that generates embeddings from input sequences. Thus, while particular embodiments and applications have been illustrated and described, it is to be understood that the described subject matter is not limited to the precise construction and components disclosed. The scope of protection should be limited only by any claims that ultimately issue.

What is claimed is:

1. A computer-implemented method of predicting a target property of a biomolecule, the method comprising:
obtaining first training data, the first training data including first biopolymer sequences and corresponding experimentally obtained data;
training a foundation model, using the first training data, to predict the experimentally obtained data from the biopolymer sequences;
adding a task-specific model to the foundation model to create a combined model;
training the combined model, using second training data, to predict the target property of biomolecules corresponding to second biopolymer sequences; and
applying the combined model to a previously unseen biopolymer sequence to generate a prediction of whether a candidate biomolecule corresponding to the previously unseen biopolymer sequence has the target property.

2. The computer-implemented method of claim 1, wherein the biopolymer is RNA.

3. The computer-implemented method of claim 1, wherein adding the task-specific model comprises removing an output head from the foundation model and replacing the output head with the task-specific model.

4. The computer-implemented method of claim 1, wherein training the combined model comprises freezing layers of the foundation model.

5. The computer-implemented method of claim 1, wherein the target property comprises secondary structure, tertiary structure, presence of a pocket with predetermined criteria, splicing activity, or whether the biomolecule will bind to a target molecule.

6. The computer-implemented method of claim 1, wherein the experimentally obtained data comprises chemical mapping data.

7. A computer-implemented method of predicting a target property of a biomolecule, the method comprising:
receiving a biopolymer sequence and an indication of the target property, the biopolymer sequence describing a biomolecule;
selecting a combined model to apply, wherein the combined model was trained by a process comprising:
obtaining first training data, the first training data including first biopolymer sequences and corresponding experimentally obtained data;
training a foundation model, using the first training data, to predict the experimentally obtained data from the biopolymer sequences;
adding a task-specific model to the foundation model to create the combined model; and
training the combined model, using second training data, to predict the target property of biomolecules corresponding to second biopolymer sequences;
applying the combined model to the biopolymer sequence to generate a prediction of whether the biomolecule has the target property; and
providing the prediction for display.

8. The computer-implemented method of claim 7, wherein the biopolymer is RNA.

9. The computer-implemented method of claim 7, wherein adding the task-specific model comprises removing an output head from the foundation model and replacing the output head with the task-specific model.

10. The computer-implemented method of claim 7, wherein training the combined model comprises freezing layers of the foundation model.

11. The computer-implemented method of claim 7, wherein the target property comprises secondary structure, tertiary structure, presence of a pocket with predetermined criteria, splicing activity, or whether the biomolecule will bind to a target molecule.

12. The computer-implemented method of claim 7, wherein the experimentally obtained data comprises chemical mapping data.

13. A non-transitory computer-readable storage medium comprising computer program code that, when executed by a computing system, causes the computing system to perform operations including:
receiving a biopolymer sequence and an indication of the target property, the biopolymer sequence describing a biomolecule;
selecting a combined model to apply, wherein the combined model was trained by a process comprising:
obtaining first training data, the first training data including first biopolymer sequences and corresponding experimentally obtained data;
training a foundation model, using the first training data, to predict the experimentally obtained data from the biopolymer sequences;
adding a task-specific model to the foundation model to create the combined model; and
training the combined model, using second training data, to predict the target property of biomolecules corresponding to second biopolymer sequences;
applying the combined model to the biopolymer sequence to generate a prediction of whether the biomolecule has the target property; and
providing the prediction for display.

14. The non-transitory computer-readable storage medium of claim 13, wherein the biopolymer is RNA.

15. The non-transitory computer-readable storage medium of claim 13, wherein adding the task-specific model comprises removing an output head from the foundation model and replacing the output head with the task-specific model.

16. The non-transitory computer-readable storage medium of claim 13, wherein training the combined model comprises freezing layers of the foundation model.

17. The non-transitory computer-readable storage medium of claim 13, wherein the target property comprises secondary structure, tertiary structure, presence of a pocket with predetermined criteria, splicing activity, or whether the biomolecule will bind to a target molecule.

18. The non-transitory computer-readable storage medium of claim 13, wherein the experimentally obtained data comprises chemical mapping data.
